(19) United States
(12) Patent Application Publication
(10) Pub. No.: US 2013/0045238 A1
(43) Pub. Date: Feb. 21, 2013

(54) EMULSIONS FOR TRANSDERMAL DELIVERY

(71) Applicants: Edwin Pei Yong Chow, Singapore (SG); Jackie Y. Ying, Singapore (SG); Shu Jun Gao, Singpore (SG)

(72) Inventors: Edwin Pei Yong Chow, Singapore (SG); Jackie Y. Ying, Singapore (SG); Shu Jun Gao, Singpore (SG)

(73) Assignee: Agency for Science, Technology and Research, Connexis (SG)

(21) Appl. No.: 13/265,445

(22) PCT Filed: Apr. 22, 2010
(86) PCT No.: PCT/US10/01194

§ 371 Date: Sep. 26, 2012

(30) Foreign Application Priority Data
Apr. 22, 2009 (SG) 2009027343

(51) Int. Cl. A61K 9/107; A61K 47/34; A61P 17/02; A61K 31/496; A61P 17/00

(52) U.S. Cl. 424/400; 514/253.08; 514/772.3

(57) ABSTRACT

The present invention generally relates to transdermal delivery and, in particular, to transdermal delivery using nanoemulsions and other emulsions. In one aspect, the present invention is directed to emulsions comprising a first, continuous phase and a second, discontinuous phase. The first phase may be an aqueous liquid and the second phase may comprise a lipid, such as isopropyl myristate. In some cases, a surfactant, such as Pluronic® L61, is used to stabilize the emulsion. Surprisingly, it has been found that such emulsions are effective at delivering pharmaceutically active agents, such as ciprofloxacin, when the formulation has a very low water content, for example, less than 30 wt % or less than 10 wt %. This is surprising because high water contents—not low water contents—are typically correlated with greater transdermal drug delivery, and thus, a low water content would have been considered to be unfavorable for facilitating transdermal drug delivery.

### RELATED APPLICATIONS

This application claims priority to Singaporean Patent Application Serial No. 2000902734-3, filed 22 Apr. 2009, entitled “Nanoemulsions for Transdermal Delivery: A New Vehicle for Dermocosmetics,” incorporated herein by reference.

### FIELD OF INVENTION

The present invention generally relates to transdermal delivery and, in particular, to transdermal delivery using nanoemulsions and other emulsions.

### BACKGROUND

There has been increased interest in recent years in the use of topical vehicle systems that could modify drug permeation through the skin. The main function of the skin is to provide a barrier to the transport of water and substances harmful to the body from entering the body. The ability of a chemical substance to penetrate the skin often depends on the composition of the substance and/or a carrier containing the substance. When a drug or cosmetic is applied to the skin, the rate of cutaneous permeation is often limited by factors such as the release of active ingredients from a carrier containing the drug or cosmetic, or the penetration of the latter through the skin. However, because such rates of transport are often slow, improvements in drug delivery techniques are still needed.

### SUMMARY OF THE INVENTION

The present invention generally relates to transdermal delivery and, in particular, to transdermal delivery using nanoemulsions and other emulsions. The subject matter of the present invention involves, in some cases, interrelated products, alternative solutions to a particular problem, and/or a plurality of different uses of one or more systems and/or articles. In some embodiments, a composition for transdermal drug delivery comprises an emulsion comprising a continuous aqueous phase and a discontinuous lipid phase comprising droplets having an average diameter of less than about 1 micrometer, the emulsion comprising a copolymer of poly(ethylene glycol) and poly(propylene glycol) having a weight percentage of at least about 40%, a lipid having a weight percentage of at least about 25%, water having a weight percentage of no more than about 10%, and a pharmaceutically active agent.

In other embodiments, a composition for transdermal drug delivery comprises an oil and water emulsion comprising a continuous phase and a discontinuous phase, the emulsion comprising droplets having an average diameter of less than about 1 micrometer, the emulsion comprising water in an amount of no more than about 10% by weight and a pharmaceutically active agent, wherein the emulsion, when positioned against mammalian skin, delivers the pharmaceutically active agent across the skin at a rate of at least about 0.2 mg/cm²/h.

In some embodiments, a composition for transdermal drug delivery comprises an emulsion comprising a continuous aqueous phase and a discontinuous lipid phase comprising droplets having an average diameter of less than about 1 micrometer, the emulsion comprising a copolymer of poly(ethylene glycol) and poly(propylene glycol) and a pharmaceutically active agent, wherein the emulsion comprises no more than about 10 wt % water.

In other embodiments, a composition comprises a copolymer of poly(ethylene glycol) and poly(propylene glycol) having a weight percentage of at least about 40%, a lipid having a weight percentage of at least about 25%, and water having a weight percentage of no more than about 10%.

In some embodiments, a method comprises providing a premix comprising a copolymer of poly(ethylene glycol) and poly(propylene glycol), a lipid, and water, wherein no more than 10 wt % of the premix is water, and producing an emulsion from the premix comprising a continuous phase and a discontinuous phase, wherein the discontinuous phase has an average droplet size of less than about 1000 nm.

In another aspect, the present invention is directed to a method of making one or more of the embodiments described herein, for example, nanoemulsions for transdermal delivery.

In another aspect, the present invention is directed to a method of using one or more of the embodiments described herein, for example, nanoemulsions for transdermal delivery.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate certain physical properties of a nanoemulsion according to one embodiment of the invention;

FIG. 2 illustrates a TEM image of a nanoemulsion according to another embodiment of the invention; and

FIG. 3 illustrates release data in yet another embodiment of the invention.

### DETAILED DESCRIPTION

The present invention generally relates to transdermal delivery and, in particular, to transdermal delivery using nanoemulsions and other emulsions. In one aspect, the present invention is directed to emulsions comprising a first, continuous phase and a second, discontinuous phase. The first phase may be an aqueous liquid and the second phase may comprise a lipid, such as isopropyl myristate. In some cases, a surfactant, such as Pluronic® L61, is used to stabilize the emulsion. Surprisingly, it has been found that such emulsions are effective at delivering pharmaceutically active agents, such as ciprofloxacin, when the formulation has a very low water content, for example, less than 30 wt % or less than 10 wt %. This is surprising because high water contents—not low water contents—are typically correlated with greater transdermal drug delivery, and thus, a low water content would have been considered to be unfavorable for facilitating transdermal drug delivery.

Higher water contents have typically been associated with increased fluidity in the lipid bilayers forming the stratum corneum, which is the upper layer of the skin, and the main barrier to transdermal drug delivery. Transdermal drug delivery formulations with high water content would have been preferred by those of ordinary skill in the art because the high water content in the formulation was thought to be necessary to increase the fluidity in the lipid bilayers forming the stratum corneum, thereby allowing greater fluxes of pharmaceutically active agents across the skin. However, as discussed herein, relatively low water contents can nevertheless be used to increase transport of pharmaceutically active agents across the skin in some cases. Accordingly, one aspect of the invention is generally directed to compositions for transdermal drug delivery having relatively low water content.

In one set of embodiments, the composition is an emulsion. An emulsion typically comprises a continuous phase and a discontinuous phase, where the discontinuous phase is present within the continuous phase as a series of discrete droplets. The discontinuous phase and the continuous phase can be stabilized in such a configuration due to the presence of one or more surfactants, which may be disposed at an interface between the continuous phase and a discontinuous phase, thereby stabilizing the two phases. In certain embodiments, the continuous phase is an aqueous phase, e.g., comprising water, a solution or a suspension containing water, or another fluid that is miscible in water, at least at ambient temperature (25° C.) and pressure (100 kPa). The discontinuous phase contained within the continuous phase may comprise a lipid, or other species that is not miscible in water at ambient temperature and pressure, as discussed below.

The droplets within the emulsion may be of any shape or size, and may be spherical, or non-spherical in some cases. The average diameter of the droplets in an emulsion is the arithmetic average of the characteristic diameter of each of the droplets, where the characteristic diameter is the diameter of a perfect sphere having the same volume as the droplet. The average diameter of the droplets may be, for example, less than about 1 mm, less than about 300 micrometers, less than about 100 micrometers, less than about 30 micrometers, less than about 10 micrometers, less than about 3 micrometers, less than about 1 micrometer, less than about 300 nm, less than about 100 nm, less than about 30 nm, or less than about 10 nm in some embodiments. Such characteristic diameters may be determined using any suitable technique known to those of ordinary skill in the art, for example, laser light scattering, small angle neutron scattering, or electron microscopy. In some embodiments, the emulsion is a “nanoemulsion,” i.e., an emulsion having an average diameter of droplets contained therein that is less than about 1 micrometer.

As mentioned, an emulsion will typically include an aqueous phase and a lipid or oil phase, where one of these phase constitutes the droplets and the other phase constitutes the continuous phase containing the droplets, i.e., the continuous phase may be the aqueous phase or the oil phase, and the discontinuous phase may be the other phase. The aqueous phase may be any phase that is miscible in water (including water itself). For example, the emulsion phase may comprise water, a solution or a suspension containing water, or another fluid which is miscible in water, at least at ambient temperature (25° C.) and pressure (100 kPa). As used herein, two fluids are immiscible, or not miscible, with each other when one is not soluble in the other to a level of at least 10% by weight at the temperature and under the conditions at which the emulsion is used, typically ambient temperature and pressure; otherwise the fluids are miscible.

Even if an aqueous phase is present, the emulsion may contain a relatively low amount of the aqueous phase. For example, the emulsion may comprise no more than about 30 wt %, no more than about 20 wt %, no more than about 10 wt %, or no more than about 5 wt % water. Under some conditions, emulsions having relatively low water contents may be useful for the transdermal of certain types of pharmaceutically active agents. This is quite unexpected because high water contents, rather than low water contents, have typically been reported as being correlated with greater transdermal drug delivery, since higher water contents increase the fluidity in the lipid bilayers forming the stratum corneum, thereby allowing greater fluxes of pharmaceutically active agents across the skin. However low water contents may also be used for transdermal drug delivery. In some cases, the high surface area provided by the emulsions droplets may allow for an increased surface area contact with the skin, thus allowing for effective transport of the pharmaceutically active agents to the skin. In some embodiments, the emulsions may aid in skin penetration of pharmaceutically active agent and an increase in the concentration of the pharmaceutically active agents in the skin may be observed. In fact, as discussed herein, such inventive emulsions can be used to deliver drugs transdermal in effective amounts, even compared to emulsions having greater amounts of water present (e.g., having at least 15 wt % water). See, e.g., Example 1.

Besides the aqueous phase, the emulsion may also contain an oil phase. However, it should be noted that, as used herein, the “oil phase” is a phase that is not miscible in water at ambient temperature and pressure, as defined above. As is understood by those of ordinary skill in the art, the “oil phase” need not have an actual oil present in it (although it can in some cases), rather the use of the term “oil phase” is used as a way of referring to the other phase that is not the phase that is miscible with water. For example, the oil phase may comprise a lipid, an oil, a fat, and/or a wax such as those commonly used in food, cosmetics, or pharmaceutical applications. These may be of natural or synthetic origin. Non-limiting examples include long chain alcohols, glyceryl esters of fatty acids or fatty esters of monohydric alcohols. These esters and alcohols can be straight or branch chained, saturated or unsaturated and the number of carbon atoms may range from C₃ to C₃₆, including all numbers within this range. Specific examples include, but are not limited to, isopropyl myristate, isopropyl palmitate, squalene, squalane, glycerol, and/or tocopheryl acetate. Other examples of lipids include fatty acids, triglycerides, phospholipids, sphingolipids, sterols, prenol lipids, saccharolipids, or polyketides.

As mentioned, the emulsion comprising the oil phase and the aqueous phase may be stabilized in a configuration with a continuous phase and a discontinuous phase due to the presence of a surfactant. For instance, the average diameter of the droplets within a stabilized emulsion may change by no more than about 10% or about 5% when the emulsion is exposed to 25° C. and 1 atm for at least about 30 days, about 60 days, or even about 90 days or longer. Typically, a surfactant has a polar “head” group and one or more nonpolar “tail” groups. The head group may be for example, a charged group or moiety, or a hydrophilic group. The nonpolar tail group may be, for example, a hydrocarbon such as a straight chain alkyl group, which optionally may contain one or more double bonds in some embodiments. Typically, when at an interface between an aqueous phase and an oil phase, the “head” or hydrophilic portion of the surfactant is in the aqueous phase, while the “tail” or hydrophobic portion of the surfactant is in the oil phase.

In certain embodiments, the surfactant is a copolymer of poly(ethylene glycol) and poly(propylene glycol). Poly(propylene glycol) is relatively hydrophobic and acts as the “tail” group, while poly(ethylene oxide) is relatively hydrophilic and acts as the “head” group of the surfactant molecules. The poly(ethylene glycol) and poly(propylene glycol) groups may be present as discrete blocks, i.e., forming a block copolymer, and any number of these blocks may be present within the copolymer. In one set of embodiments, the poly(ethylene glycol) and poly(propylene glycol) blocks are present as nonionic triblock copolymers formed from a poly(propylene oxide) center with two flanking poly(ethylene oxide) blocks. Examples of such copolymers include poloxamers such as those sold under the trade name Pluronic®.

In one embodiment, the surfactant is Pluronic® L61. In Pluorinc® formulations, the first letter denotes its physical form at room temperature (L=liquid, P=paste, F=flake (solid)) followed by two or three digits. The first digit (two digits in a three-digit number) in the numerical designation, multiplied by 300, indicates the approximate molecular weight of the poly(propylene oxide) blocks; and the last digit×10 gives the percentage poly(ethylene glycol) content (e.g., Pluronic® L61 is a molecule having a poly(propylene oxide) molecular weight of about 1,800 g/mol and an approximately 10% poly(ethylene glycol) content). (It should be understood, of course, that in such surfactant formulations, these numbers represent approximations rather than being exact; in reality, there may a distribution of molecules present within the surfactant formulation, and/or the actual average molecular weight may not necessarily be exact.) Other suitable Pluronic surfactants that could be used in various embodiments of the present invention include, but are not limited to, Pluronic® L64, Pluronic® L81, Pluronic® L101, Pluronic® 121, Pluronic® F68, Pluronic® 108, Pluronic® L62, or Pluronic® F 127.

Emulsions such as those described above may be used for transdermal drug delivery applications, where the emulsion is administered to the skin of a subject, and a pharmaceutically active agent contained within the emulsion passes across the skin into the subject, where the agent may be locally or systemically distributed, depending on the agent.

In some cases, fluxes of the pharmaceutically active agent of at least about 0.1 mg/cm²/h may be achieved, and in some cases, the flux may be at least about 0.2 mg/cm²/h, at least about 0.3 mg/cm²/h, at least about 0.5 mg/cm²/h, at least about 1 mg/cm²/h, at least about 3 mg/cm²/h, or even more. The subject is usually human, although non-human subjects may be used in certain instances, for instance, other mammals such as a dog, a cat, a horse, a rabbit, a cow, a pig, a sheep, a goat, a rat, a mouse, a guinea pig, a hamster, a primate (e.g., a monkey, a chimpanzee, a baboon, an ape, a gorilla, etc.), or the like. The pharmaceutically active agent may be any suitable agent that beneficially may be administered to a subject, e.g., to the skin of the subject. In one set of embodiments, the pharmaceutically active agent is substantially hydrophobic, i.e., when prepared in an emulsion as described herein, the pharmaceutically active agent is found in a higher concentration in the oil phase of the emulsion, relative to the aqueous phase of the emulsion. For example, if the emulsion comprises isopropyl myristate as the oil phase, in some cases, the pharmaceutically active agent may have a greater solubility in isopropyl myristate than in water.

In one set of embodiments, the pharmaceutically active agent is an antibiotic. Examples of antibiotics include quinolones or fluoroquinolones, such as ciprofloxacin. In some cases, the pharmaceutically active agent is an antineoplastic agent, an immunostimulant agent, an immunosuppressant agent, an antiviral agent, an antibacterial agent, an antifungal agent, an antiparasitic agent, a pharmacological active agent, a fat-soluble cosmetic active substances, or the like. Other non-limiting examples of pharmaceutically active agents include phytochemical plant-derived or microbial extracts or synthesised peptides, antioxidant agents (e.g., stilbenes and derivatives such as Resveratrol, Pterostilbene, alpha hydroxy acids), derma fillers, Botox, peptides or proteins (e.g., derived from adult adipose or placenta stem cells, which may have regenerative effects), and the like.

As mentioned, the emulsion may contain a very low amount of water. For example, the emulsion may comprise no more than about 30 wt %, no more than about 20 wt %, no more than about 10 wt %, or no more than about 5 wt % water. In some cases, most or all of the remainder of the emulsion comprises surfactant, an oil phase, and a pharmaceutically active agent. For instance, at least about 40% (by weight) of the emulsion may be surfactant and at least about 25% (by weigh) of the emulsion may be the oil phase. In some embodiments, a surfactant, such as a copolymer of poly(ethylene glycol) and poly(propylene glycol), may be present in the emulsion at a concentration of at least about 40% (by weight).

In some cases, the percentage may be higher, e.g., at least about 45%, at least about 50%, at least about 55%, at least about 60%, at least about 70%, at least about 75%, at least about 80%, at least about 85%, or at least about 90%. In some cases, the surfactant may comprise no more than about 90% (by weight), no more than about 85%, no more than about 80%, no more than about 75%, no more than about 70%, no more than about 65%, no more than about 60%, no more than about 55%, no more than about 50%, or no more than about 45% of the emulsion. In certain embodiments, a lipid, such as isopropyl myristate, may be present in the emulsion at a concentration of at least about 25% (by weight).

In certain cases, the percentage may be higher, e.g., at least about 30%, at least about 35%, at least about 40%, at least about 45%, at least about 50%, at least about 55%, at least about 60%, at least about 70%, at least about 75%, at least about 80%, at least about 85%, or at least about 90%. In certain instances, the lipid may comprise no more than about 90% (by weight), no more than about 85%, no more than about 80%, no more than about 75%, no more than about 70%, no more than about 65%, no more than about 60%, no more than about 55%, no more than about 50%, or no more than about 45% of the emulsion.

In some embodiments, at least about 80% (by weight) of the emulsion may be water, surfactant, oil phase, or pharmaceutically active agent, and in some cases, the total weight percentage of these may be higher, e.g., at least about 85%, at least about 90%, or at least about 95%. The balance of the emulsion may include other compounds, for example, one or more salts, buffers, excipients, chelating agents, fillers, antioxidants, antimicrobials, preservatives, binding agents, silicas, stabilizers, dispersion media, release-retarding agents, etc.

The composition may take any of a wide variety of forms suitable for transdermal drug delivery, for example, a paste, a lotion, a cream, or the like that is applied to the surface of the skin. In some cases, the composition may be applied as part of a “patch” that is adhered to the skin, where the patch typically including a backing, and optionally an adhesive, in addition to the composition.

Another aspect of the present invention is directed to systems and methods for making emulsions such as those described herein. A variety of such techniques can be used. For example, an emulsion may be formed by shaking, stirring, homogenizing, or spraying a first phase and a second phase (e.g., an oil phase and an aqueous phase) such that one of the phases becomes dispersed in the other, i.e., such that one phase becomes discontinuous and forms droplets contained within the other phase. The first phase and the second phase, prior to mixing, may be a premix, and may comprise one or more of a surfactant, a lipid or oil phase, water or another aqueous phase, and a pharmaceutically active agent.

In one set of embodiments, a premix may be formed having substantially the same composition as the desired emulsion, e.g., as previously discussed. The premix may comprise a first phase and a second phase, e.g., an aqueous phase and an oil phase. The premix may then be exposed to shear forces sufficient to produce an emulsion comprising a continuous phase and a discontinuous phase. Depending on the amount of shear, the size of the droplets in the discontinuous phase may be controlled, for example, such that droplets having an average diameter of the droplets contained therein of less than about 1 mm are formed, or any other droplet sizes as discussed herein. For instance, at sufficient shear, a nanoemulsion may be formed.

In some cases, the shear force may be varied by varying the rpm (revolutions per minute) applied to the premix (e.g., using a rotor, a mechanical mixer, etc.). In some cases, the rotations per minute may be between about 500 and about 5000 rpm, between about 1000 and about 4000 rpm, between about 1500 and about 3500 rpm, between about 2000 and about 3200 rpm, etc. The premix may be exposure to the shear force for any period of time sufficient to form the desired nanoemulsion. In some cases, the premix may be exposed to the shear force for a period of time between about 1 minute and about 60 minutes, between about 1 minute and about 30 minutes, between about 1 minute and about 20 minutes, between about 5 minutes and about 15 minutes, between about 5 minutes and about 10 minutes, between about 10 minutes and about 15 minutes, etc.

In some embodiments, a nanoemulsion (e.g., and oil-in-water nanoemulsion) may be prepared by at least partially dissolving and/or suspending at least one surface-active agent in a first phase (e.g., an oil phase), adding a second phase (e.g., an aqueous phase) under vigorous agitation, until complete homogenization. In some cases, the agitation may be provided by a vortex mixer, a stirrer (e.g., magnetic stirrer), etc.

Such emulsions may be administered to a subject, in yet another aspect of the present invention. The composition comprising the emulsion may be administered to the skin of the subject, at any suitable region or area, depending on the application. For example, if an antibiotic is used, the composition may be applied to a site of infection, to a wound site, or to another convenient area of the body (e.g., for systemic circulation).

In one set of embodiments, the composition is administered to a subject to treat a wound. In another set of embodiments, the composition is administered to a subject to treat dry skin or xeroderma. Xeroderma occurs most commonly on the scalp, lower legs, arms, the knuckles, the sides of the abdomen and thighs. Symptoms most associated with xeroderma are scaling (the visible peeling of the outer skin layer), itching, or cracks in the skin. In still another set of embodiments, the composition is administered to a subject to treat an age-related skin disease, such as wrinkles, sagging skin, pigmentation or uneven skin color, or a loss of strength or elasticity of the skin.

In certain aspects, a composition of the invention can be combined with a suitable pharmaceutically acceptable carrier, for example, as incorporated into a liposome, incorporated into a polymer release system, or suspended in a liquid. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. The carrier may be either soluble or insoluble, depending on the application. Those skilled in the art will know of other suitable carriers, or will be able to ascertain such, using only routine experimentation.

In some embodiments, the compositions of the invention include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

Another aspect provides a method of administering any composition of the present invention to a subject, e.g., as a drug or as a cosmetic. When administered as a treatment for a disease, the compositions of the invention are applied in a therapeutically effective, pharmaceutically acceptable amount as a pharmaceutically acceptable formulation. As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result. The terms “treat,” “treated,” “treating,” and the like, when used herein with respect to a disease, refer to administration of the inventive compositions to a subject which may increase the resistance of the subject to development or further development of the disease, to administration of the composition after the subject has developed the disease in order to eliminate or at least control development of the disease, and/or to reduce the severity of symptoms caused by the disease. When administered to a subject, effective amounts will depend on the particular condition being treated and the desired outcome. A therapeutically effective dose may be determined by those of ordinary skill in the art, for instance, employing factors such as those further described below and using no more than routine experimentation.

The dose of the composition to the subject may be such that a therapeutically effective amount of the composition reaches the active site of the composition within the subject. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject.

In certain embodiments of the invention, the administration of the composition of the invention may be designed so as to result in exposures to the composition over a certain time period, for example, hours, days, weeks, months or years. This may be accomplished, for example, by repeated administrations of a composition of the invention, or by a sustained or controlled release delivery system in which the composition is delivered over a prolonged period without repeated administrations. Maintaining a substantially constant concentration of the composition may be preferred in some cases.

The present invention also provides, in other aspects, any of the above-mentioned compositions in kits, optionally including instructions for use of the composition, e.g., by any suitable technique as previously described. The invention also involves promotion of a composition as described herein for any suitable use, e.g., for the treatment of a disease, for the application of a cosmetic, or the like. As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with use of the composition. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention and the instructions in any form that are provided in connection with the composition in a manner such that a clinical professional will clearly recognize that the instructions are to be associated with the specific composition.

The kits described herein may also contain one or more containers, which may contain the inventive composition and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the compound and the mode of use or administration. The solvent will depend on the compound and the mode of use or administration.

Singaporean Patent Application Serial No. 2000902734-3, filed 22 Apr. 2009, entitled “Nanoemulsions for Transdermal Delivery: A New Vehicle for Dermocosmetics,” is incorporated herein by reference.

The following examples are intended to illustrate certain embodiments of the present invention, but do not exemplify the full scope of the invention.

### EXAMPLE 1

This example illustrates the potential of nanoemulsion systems in transdermal delivery of ciprofloxacin using non-irritating, pharmaceutically acceptable ingredients without employing additional permeation enhancers, in accordance with certain embodiments of the invention. This is because the excipients of nanoemulsions themselves acted as permeation enhancers.

A nanoemulsion was prepared comprising Pluronic® L61, isopropyl myristate, ciprofloxacin, and water. Poly(ethylene glycol)-block-poly(propylene glycol)-block-poly(ethylene glycol) Pluronic® L61 was a gift from BASF. Isopropyl myristate (IPM) and ciprofloxacin was purchased from Sigma Aldrich. All reagents and solvents were used as received. Water was purified by a Milli-Q water purification system.

The region of the nanoemulsion comprising Pluronic L61, IPM, and water was determined systematically by titrating water to various compositions of Pluronic L61 and

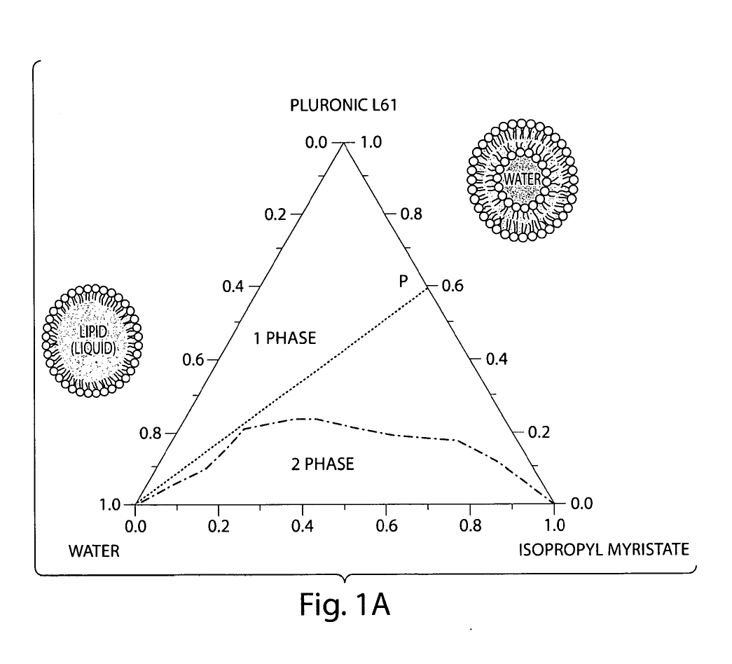

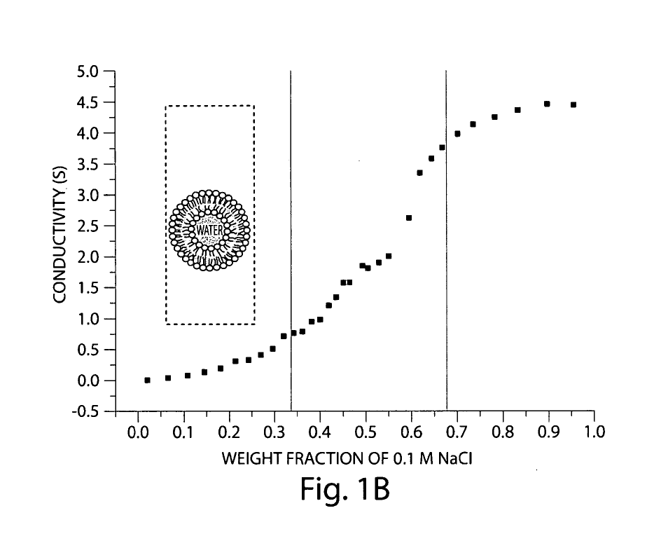

IPM in a screw-capped test tube. Each sample was vortex-mixed and allowed to equilibrate in a temperature-controlled environment at 25° C. Each sample was then studied to determine its clearness (transparency) or turbidity. These clear/turbid points were used to establish phase boundaries of the nanoemulsion in a phase diagram (see FIG. 1A). This figure shows a phase diagram of Pluronic L61, isopropyl myristate and water (in wt %), indicating the nanoemulsion region (unshaded area). The nanoemulsions could be further classified as oil-in-water (O/W), bicontinuous, or water-in-oil (W/O), according to conductivity measurements using a conductivity meter.

From the phase diagram, different formulas were selected from the nanoemulsion region so that drugs could be incorporated into the aqueous phase. 0.25 mg of ciprofloxacin (chosen arbitrarily) was used in all the selected formulations in this example. It was dissolved in the aqueous phase of the nanoemulsion.

Selected formulations were subjected to thermodynamic stability tests. The formulations were centrifuged at 822 g (1 g=standard gravitational acceleration) for 30 min. The formulations that did not show any phase separations were taken through six cycles of cooling (at a refrigerator temperature of 4° C.) and heating (45° C.); they were kept at each temperature for at least 48 h. The formulations that were stable at these temperatures were then subjected to three freeze-thaw cycles between −21° C. and 25° C. The formulations that passed these thermodynamic stability tests were chosen for further studies.

The morphology and structure of the nanoemulsions were studied using transmission electron microscopy (TEM) (FEI Tecnai G2 F20 electron microscope, 200 kV), with the software package for automated electron tomography. A drop of the nanoemulsion was directly deposited on the film grid and observed by TEM after drying.

The droplet size distribution of the nanoemulsions were determined by photon correlation spectroscopy (Zetasizer 1000 HS, Malvern Instruments, Worchestershire, UK). Light scattering was conducted at 25° C. at a 90° angle. The refractive indices of ciprofloxacin-loaded formulations were determined using an Abbe-type refractometer (Nirmal International).

In vitro skin permeation studies were performed on a Franz diffusion cell with an effective diffusional area of 0.636 cm² and 4 mL of receiver chamber capacity using porcine abdominal skin. An automated transdermal diffusion cell sampling system (SFDC6, Logan Instruments, NJ, USA) was used for these studies. Full-thickness porcine skin was excised from the abdominal region, and hair was removed with an electric clipper. The subcutaneous tissue was removed surgically, and the dermis side was wiped with isopropanol to remove the adhering fat. The cleaned skin was washed with deionized water, and stored in a deep freezer at −21° C. until further use. The skin was then brought to room temperature, and mounted between the donor and receiver compartment of the Franz diffusion cell, whereby the stratum corneum side faced the donor compartment and the dermal side faced the receiver compartment.

Initially, the donor compartment was empty and the receiver chamber was filled with phosphate-buffered saline (PBS) (pH 7.4). The receiver fluid was stirred with a magnetic rotor at 600 rpm. The assembled apparatus was placed in the transdermal permeation apparatus and kept at 32±1° C. The PBS was replaced completely every 30 min to stabilize the skin. It was found that the receiver fluid showed negligible absorbance after 4.5 h, indicating complete stabilization of the skin. After complete stabilization of the skin, 1 mL of the nanoemulsion formulation (with 0.25 mg/mL ciprofloxacin) was placed in each donor compartment and sealed with paraffin film to provide occlusive conditions. Samples were withdrawn at regular intervals, filtered through a 0.45-micrometer membrane filter, and analyzed for drug content by UV spectrophotometer at the maximum wavelength (λₘₐₓ, lambda-max). The skin permeation profiles of different nanoemulsion formulations were then compared.

Sin irritation tests were conducted on six SD rats. The animals were kept under standard laboratory conditions at 25±1° C. They were housed in polypropylene cages with access to a standard laboratory diet (Lipton feed, Mumbai, India) and water ad libitum. The test article used to conduct the skin irritation tests was a filter paper patch (0.5 cm in diameter) saturated with different nanoemulsions. The backs of the animals were clipped free of fur with an electric clipper, and de-hair cream was applied at least 24 h before sample application. Each rat received six test samples. The patches were backed with plastic, and covered with a non-reactive tape; the entire test site was wrapped with a binder. The animals were then returned to their cages. The test sites were examined 24 h, 72 h, 1 week, 2 weeks, 3 weeks, and 4 weeks after sample application for dermal reactions, in accordance with the FHSA-recommended Draize scoring criteria (Federal Hazardous Substances Act).

The excipients were selected to be pharmaceutically acceptable, non-irritating and non-sensitizing to the skin, and in some cases fall into the GRAS (generally regarded as safe) category. High solubility of the drug in the aqueous phase was another important criterion, since that would help the nanoemulsion to maintain the drug in the solubilized form. Safety is a major factor in surfactant selection since a large amount of surfactants may cause skin irritation. Non-ionic surfactants are less toxic than ionic surfactants. Another important criterion for surfactants is that the hydrophilic lipophilic balance (HLB) for forming W/O nanoemulsion was selected to be less than 10. The right choice of low HLB surfactants would lead to the formation of a stable nanoemulsion formulation.

In this example, Pluronic L61 was selected as a surfactant. Pluronic L61 has an HLB value of 1 to 7. Transient negative interfacial tension and fluid interfacial film were achieved by the use of single surfactant forming nanoemulsions over a wide range of compositions. Ciprofloxacin is a lipophilic drug, and its physicochemical properties suggested that it had good potential for transdermal drug delivery. Therefore, in the present example, different nanoemulsions were prepared for the transdermal delivery of ciprofloxacin.

Constructing phase diagrams can be time-consuming in some cases, particularly when the aim is to accurately delineate a phase boundary. Care was taken to ensure that observations were not made on metastable systems, whereby although the free energy required to form an emulsion is very low, the formation is thermodynamically spontaneous. Ternary phase diagrams have been constructed separately for each surfactant-to-oil ratio, so that different nanoemulsion regions could be identified for the optimization of nanoemulsion formulations.

FIG. 1A shows the phase behavior of the nanoemulsion region of a system of Pluronic L61, IPM, and water. The one-phase region represents a range of compositions that could be selected to form transparent nanoemulsions. A nanoemulsion can be formed with an aqueous content of 20 wt % to 50 wt %. The change in the conductivity of nanoemulsions with the aqueous content along the P-line (dotted line) is illustrated in FIG. 1B. The low conductivity for systems containing less than 30 wt % of aqueous content was attributed to the formation of W/O nanoemulsion with aqueous droplets dispersed in a continuous oil phase. The sharp increase in conductivity for the systems containing greater than 30 wt % of aqueous content was associated with the presence of numerous interconnected conducting channels, which was characteristic for the bicontinuous phase. The boundary between the bicontinuous nanoemulsion and O/W nanoemulsion was not established in this example since this example was focused on nanoemulsions containing less than 30 wt % of aqueous content. Nanoemulsions are relatively thermodynamically stable systems, and can be formed at particular concentrations of oil, surfactant and water. They would not be subjected to phase separation, creaming, or cracking. Thermal stability differentiates nanoemulsions from emulsions that have kinetic stability, but eventually only kinetic stability formulations will undergo phase separation. Thus, the formulations were tested for their thermodynamic stability via centrifugation, heating-cooling cycles, and freeze-thaw cycles. Only formulations that survived the thermodynamic stability tests were selected for further studies (see Table 1).

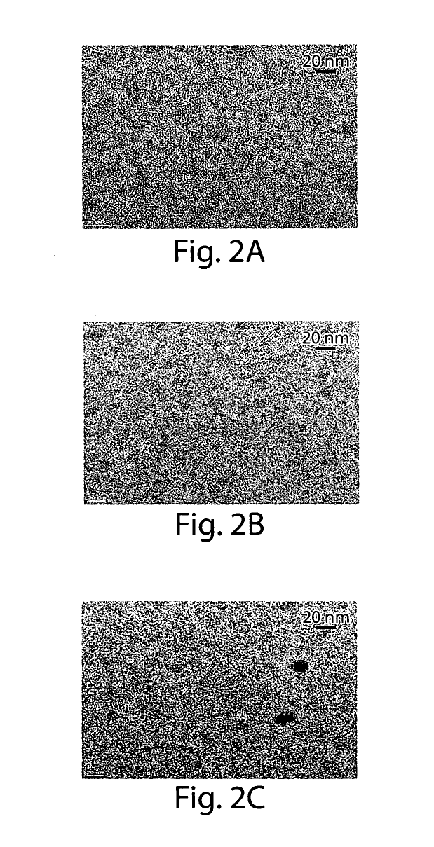

In a positive TEM image, the nanoemulsion appeared dark and the surroundings were bright (FIG. 2). The droplet sizes were measured by TEM. These dimensions were in agreement with the droplet size distributions characterized with photon correlation spectroscopy (Table 1). FIGS. 2A, 2B, and 2C are TEM images of the NE-1, NE-2, and NE-3 nanoemulsions, respectively.

TABLE 1
Composition, droplet size and polydispersity of nanoemulsions (n = 3).

| Nanoemulsions | | | |
|---|---|---|---|
| Composition and | | | |
| Characteristics | NE-1 | NE-2 | NE-3 |
| Pluronic L61 (g) | 1.2 | 1.2 | 1.2 |
| Isopropyl myristate (g) | 0.8 | 0.8 | 0.8 |
| Water (g) | 0.105 | 0.35 | 0.50 |
| | (5 wt %) | (15 wt %) | (20 wt %) |
| Droplet Size (nm) | 14.1 ± 1.2 | 23.5 ± 3.0 | 24.9 ± 3.2 |
| (Mean ± SD) | | | |
| Polydispersity | 0.035 | 0.063 | 0.077 |
| Refractive Index ± SD | 1.401 ± 0.007 | 1.409 ± 0.009 | 1.403 ± 0.012 |

The droplet size increased with increasing water content in the nanoemulsions (Table 1). NE-1, which contained 5 wt % of water, had the smallest droplet size of 14.1±1.2 nm. NE-3, which has 20 wt % of water, had the largest droplet size of 24.9 +3.2 nm. All of the formulations had droplet sizes in the nanometer regime with low polydispersity values, indicating uniformity of droplet size within each formulation. The mean refractive indices of the drug-loaded formulations were not significantly different. Thus, the nanoemulsion formulations were not only thermodynamically stable, but also chemically stable and remained isotropic, i.e. there were no interactions between the nanoemulsion excipients and the drug.

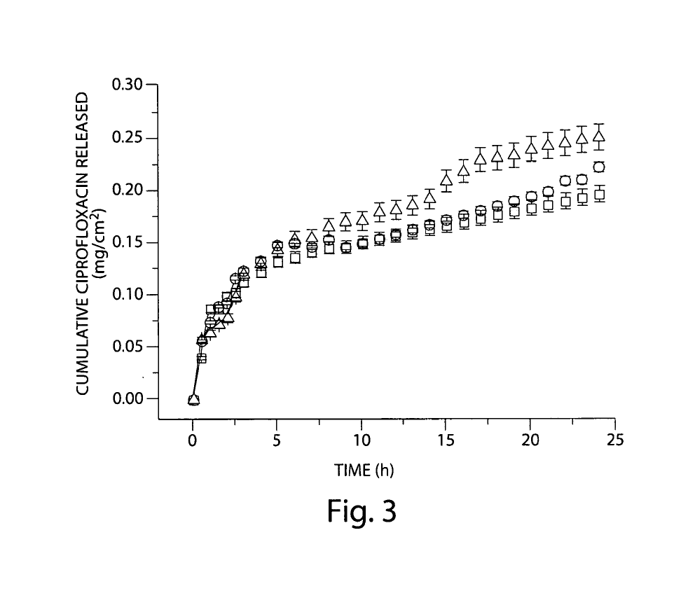

In vitro skin permeation studies were performed to compare the drug release from different nanoemulsions, each containing the same quantity (0.25 mg) of ciprofloxacin. In vitro skin permeation was highest in NE-1. This could be attributed to its smallest droplet size. These data are shown in FIG. 3 for NE-1 (triangles), NE-2 (circles), and NE-3 (squares) nanoemulsions.

A skin irritation test was performed to confirm the safety of selected nanoemulsion formulations. According to the Draize scoring criteria, a value of 0 to 4 would indicate that the erythema and eschar formation is generally not an irritant to human skin. The mean skin irritation scores for all three nanoemulsions were 0 (Table 2), confirming that these formulations were safe for use in transdermal drug delivery. No irritation was observed on the skin of the rats.

TABLE 2
Draize evaluation of dermal reaction (skin score card) for nanoemulsions.

| Sample | Reaction | 24 h | 72 h | 1 week | 2 weeks | 3 weeks | 4 weeks |
|---|---|---|---|---|---|---|---|
| NE-1 | Erythema | 0 | 0 | 0 | 0 | 0 | 0 |
| | Edema | 0 | 0 | 0 | 0 | 0 | 0 |
| NE-2 | Erythema | 0 | 0 | 0 | 0 | 0 | 0 |
| | Edema | 0 | 0 | 0 | 0 | 0 | 0 |
| NE-3 | Erythema | 0 | 0 | 0 | 0 | 0 | 0 |
| | Edema | 0 | 0 | 0 | 0 | 0 | 0 |

In summary, this example illustrates nanoemulsion systems with Pluronic L61, isopropyl myristate and water. These nanoemulsions demonstrated a high degree of stability. Their droplet size did not change over a period of at least 3 months. The nanoemulsion containing 5 wt % of water showed a higher permeation rate than those containing 15 wt % and 20 wt % of water. The skin irritation study indicated that the nanoemulsion formulations were safe for use in transdermal drug delivery. These nanoemulsions could be formulated into natural skin care lotion, cream, or serum for direct application in consumer products.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for transdermal drug delivery, the composition comprising:
an emulsion comprising a continuous aqueous phase and a discontinuous lipid phase comprising droplets having an average diameter of less than about 1 micrometer, the emulsion comprising a copolymer of poly(ethylene glycol) and poly(propylene glycol) having a weight percentage of at least about 40%, a lipid having a weight percentage of at least about 25%, water having a weight percentage of no more than about 10%, and a pharmaceutically active agent.

2. A method, comprising:
administering the composition of claim 1 to the skin of a subject.

3. A composition for transdermal drug delivery, the composition comprising:
an oil and water emulsion comprising a continuous phase and a discontinuous phase, the emulsion comprising droplets having an average diameter of less than about 1 micrometer, the emulsion comprising water in an amount of no more than about 10% by weight and a pharmaceutically active agent, wherein the emulsion, when positioned against mammalian skin, delivers the pharmaceutically active agent across the skin at a rate of at least about 0.2 mg/cm²/h.

4. A composition for transdermal drug delivery, the composition comprising:
an emulsion comprising a continuous aqueous phase and a discontinuous lipid phase comprising droplets having an average diameter of less than about 1 micrometer, the emulsion comprising a copolymer of poly(ethylene glycol) and poly(propylene glycol) and a pharmaceutically active agent, wherein the emulsion comprises no more than about 10 wt % water.

5. The composition of claim 4, wherein the pharmaceutically active agent is ciprofloxacin.

6. The composition of claim 4, wherein the average diameter of the droplets is less than about 100 nm.

7. The composition of claim 4, wherein the lipid is isopropyl myristate.

8. The composition of claim 4, wherein the copolymer of poly(ethylene glycol) and poly(propylene glycol) is a triblock copolymer.

9. The composition of claim 8, wherein the triblock copolymer comprises a central block of poly(propylene oxide) and two outer blocks of poly(ethylene oxide).

10. The composition of claim 9, wherein the poly(propylene oxide) has a molecular weight of about 1800 g/mol and the copolymer has about 10 wt % poly(ethylene oxide).

11. The composition of claim 4, wherein the copolymer of poly(ethylene glycol) and poly(propylene glycol) is disposed at an interface between the aqueous phase and the liquid phase.

12. The composition of claim 4, wherein the composition is a cream.

13. The composition of claim 4, wherein the composition is a lotion.

14. The composition of claim 4, wherein the average diameter of the droplets changes by no more than about 10% when the emulsion is exposed to 25° C. and 1 atm for at least about 30 days.

15. The composition of claim 4, wherein the emulsion comprises no more than about 5 wt % water.

16. A method, comprising:
administering the composition of claim 4 to the skin of a subject.

17. The method of claim 16, wherein the subject has dry skin.

18. The method of claim 16, comprising administering the composition to a wound on the skin of the subject.

19. The method of claim 16, wherein the subject has an age-related skin disease.

20. The method of claim 16, wherein the subject is human.

21. A composition, comprising:
a copolymer of poly(ethylene glycol) and poly(propylene glycol) having a weight percentage of at least about 40%;
a lipid having a weight percentage of at least about 25%; and
water having a weight percentage of no more than about 10%.

22. The composition of claim 21, wherein the copolymer of poly(ethylene glycol) and poly(propylene glycol), the lipid, and water forms an emulsion.

23. The composition of claim 21, further comprising a pharmaceutically active agent.

24. The composition of claim 21, further comprising ciprofloxacin.

25. The composition of claim 21, wherein the copolymer of poly(ethylene glycol) and poly(propylene glycol) has a weight percentage of at least about 50%.

26. The composition of claim 21, wherein the copolymer of poly(ethylene glycol) and poly(propylene glycol) has a weight percentage of between about 55% and about 60%.

27. The composition of claim 21, wherein the lipid is isopropyl myristate.

28. The composition of claim 21, wherein lipid has a weight percentage of between about 30% and about 50%.

29. The composition of claim 21, wherein lipid has a weight percentage of between about 35% and about 40%.

30. The composition of claim 21, wherein water has a weight percentage of no more than about 5%.

31. A method, comprising:
administering the composition of claim 21 to the skin of a subject.

32. A method, comprising:
providing a premix comprising a copolymer of poly(ethylene glycol) and poly(propylene glycol), a lipid, and water, wherein no more than 10 wt % of the premix is water; and
producing an emulsion from the premix comprising a continuous phase and a discontinuous phase, wherein the discontinuous phase has an average droplet size of less than about 1000 nm.
